(19) United States
(12) Patent Application Publication
(10) Pub. No.: US 2010/0228074 A1
(43) Pub. Date: Sep. 9, 2010

(54) Therapeutic and Directionally Dosed Implants

(75) Inventors: Christopher D. Drobnik, Wauconda, IL (US); Michael W. Drobnik, Downers Grove, IL (US)

Correspondence Address: BRIAN M. BURN, ESQ.;C. R. BARD UROLOGICAL DIVISION, P.O. BOX 52050, c/o PORTFOLIOIP, MINNEAPOLIS, MN, 55402, US

(73) Assignee: C.R. Bard, Inc., Murray Hill, NJ (US)

(21) Appl. No.: 12/377,480

(22) PCT Filed: Aug. 24, 2007
(86) PCT No.: PCT/US07/76736

§ 371 Date: Feb. 13, 2009

Related U.S. Application Data
(60) Provisional application No. 60/832,555, filed on Jul. 21, 2006.

(51) Int. Cl. A61M 36/12

(52) U.S. Cl. 600/7

(57) ABSTRACT

The present disclosure relates to an implant comprising at least one sheet of a biocompatible material, at least one shielding apparatus fixed to the biological material, and at least one radioactive seed partially disposed in the shielding apparatus. The disclosure also relates to a method for treating a patient, comprising fixing to the tissue of the patient at least one implant comprising at least one sheet of a biocompatible material, at least one shielding apparatus fixed to the biological material, and at least one radioactive seed partially disposed in the shielding apparatus.

This application is claims benefit of priority to U.S. Provisional Application No. 60/823,555, filed Aug. 25, 2006, and entitled “Radioactive Sources in Two-Dimensional Array” (the disclosure of which is incorporated herein by reference in its entirety).

### BACKGROUND

Bodily cancers are commonly treated using radiation therapy. Radiation therapy employs high energy radiation to kill cancer cells. One type of radiation therapy is brachytherapy, in which a source of radiation is in direct contact with an afflicted tissue. A common brachytherapy treatment, transperineal seed implantation, involves placing radioactive seeds in the prostate gland to kill prostate gland cancer cells. A physician employs tools such as ultrasound, computerized tomography (“CT”) scans, and X-ray images in concert with dose-planning computer software programs to evaluate the medical condition of a patient. The physician constructs an optimal treatment plan to evenly distribute radiation throughout the afflicted tissue. Radioactive seeds of discrete radioactive strengths are inserted through multiple implantation needles at positions in the prostate gland corresponding to the treatment plan. Multiple implantation needles are required to insert the radioactive seeds into multiple locations in the afflicted tissue, with each needle containing a specified arrangement of the radioactive seeds.

Although brachytherapy is perhaps most often effected by needle implantation, other implantation methods have also been used. One example is a configuration employing seeds and sutures. To make such a configuration, the physicians have utilized a seed product (e.g., Seed in Carrier, manufactured by Oncura) that consists of seeds disposed within a suture material. The sutures are weaved through a piece of bioabsorbable fabric to yield a planar array of seeds. This array is then used to irradiate a tumor bed, most commonly following a lung resection, by sewing the array to the wall of the tumor bed.

Gross surgical removal of tumorous tissue can leave behind traces of cancerous tissue which can result in metastasis or recurrence of the tumor. Thus, the site of removal of a tumor is often treated postoperatively in an attempt to destroy any such diseased tissue left behind by the surgery. Conventional techniques for treating the site of surgical removal of a tumor include post-operative administration of radiation, chemotherapy, and/or heat. Another method is disclosed in U.S. Pat. No. 5,030,195, the disclosure of which is incorporated by reference herein. In accordance with that disclosure, seeds can be threaded into a mesh, and the mesh embedded in a nonabsorbable silicone compound. Once the exact location and extent of a tumor is determined, the tumor is removed, and the mesh/silicone material is embedded in a region where residual tumor cells may exist.

Proper seed placement and seed retention at the implantation site strongly influence the success or failure of a brachytherapy procedure. As described above, seed implantation devices may contain a plurality of seeds that may be separated by spacers. Prior implantation devices and methods do not reliably maintain proper seed spacing during and after implantation. Therefore, a device and/or method of reliably maintaining proper seed spacing during and after implantation would be of great benefit to brachytherapy patients.

Loose seeds implanted in the prostate, especially those that are extra-capsular (located outside the capsule of the prostate), may possibly migrate within the patient. Because extra-capsular tissue is less dense than tissue within the capsule of, e.g., the prostate, prior brachytherapy seed implantation devices and methods cannot effectively maintain the location of seeds in the extra-capsular material. These seeds may migrate and fail to provide radiation where needed. Migrating radioactive seeds not only fail to provide needed radiation therapy at the treatment site, but may cause damage to other radiation-sensitive areas of the body. Therefore, a device and/or method of preventing migration of radioactive seeds in tissues and/or fluids of varying densities and consistencies would be of great benefit to brachytherapy patients.

In view of the above, it would be desirable to have an implant, whether standard or custom, which is capable of delivering radiation to a patent in need thereof without the above-mentioned disadvantages. The present disclosure provides the ability to present the seeds to the physician ready-made on a sheet of material, rather than only supplying them loose or pre-made into simple “lines.” This can provide a more usable product that is more amenable to placement on the exterior portion of a tumor, along the suture line of a resected tumor, as well as enabling placement of seeds within the “hole” created by the excision of a tumor in order to better treat the microscopic disease located in the non-excised tumor margins. The present disclosure provides several innovations that can be incorporated to allow ease of manufacture, ease of handling, flexibility of deployment, dose delivery and reliability, etc.

### SUMMARY

According to one aspect of the present disclosure, there is provided an implant comprising at least one sheet of a biocompatible material, at least one shielding apparatus fixed to the biological material, and at least one radioactive seed partially disposed in the shielding apparatus.

According to another aspect of the present disclosure, there is provided a method for treating a patient, comprising fixing to the tissue of the patient at least one implant comprising at least two sheets of a biocompatible material, and at least one radioactive seed disposed between said sheets of material.

According to yet another aspect of the present disclosure, there is provided a method for treating a patient, comprising surgically excising at least a portion of a tumor from surrounding tissue, and providing at least one implant at the locus of said surrounding tissue, wherein the implant comprises at least two sheets of a biocompatible material, and at least one radioactive seed disposed between said sheets of material.

### BRIEF DESCRIPTION OF THE DRAWINGS

The disclosed embodiments can be better understood with reference to the following drawing. The components in the drawing are not necessarily to scale.

FIG. 1, according to various embodiments, is an illustration of an implant in accordance with the present disclosure.

FIG. 2, according to various embodiments, is a detailed illustration of an implant having radioactive seeds in accordance with the present disclosure.

FIG. 3, according to various embodiments, is an overall illustration of an implant having radioactive seeds in accordance with the present disclosure.

FIG. 4, according to various embodiments, is a detailed illustration of an alternative embodiment of an implant having radioactive seeds in accordance with the present disclosure.

FIG. 5, according to various embodiments, is an overall illustration of an alternative embodiment of an implant having radioactive seeds in accordance with the present disclosure.

FIG. 6, according to various embodiments, is an overall illustration of an implant having radioactive seeds in a predetermined pattern in accordance with the present disclosure.

FIG. 7, according to various embodiments, is an overall illustration of an implant having radioactive seeds in another predetermined pattern in accordance with the present disclosure.

FIG. 8, according to various embodiments, is an overall illustration of an implant having radioactive seeds disposed in a shielding material that is attached to a sheet of biocompatible material.

### DESCRIPTION

According to various embodiments, the present disclosure relates to an implant comprising at least one seed in a carrier. For example, the implant comprises a plurality of seeds. The implant can comprise an array, for example a planar array, of seeds. The seeds can be disposed in an array on the material, based on horizontal and vertical separation of the seeds. The implant can also comprise seeds arranged in a three dimensional construction. For example, the seeds can be disposed in a flexible mass (such as a sphere) of mesh that could be collapsed, inserted into a body cavity and allowed to naturally expand to fill the cavity. According to various embodiments, the mass could be flexible enough to conform to an area that would not be perfectly spherical. According to various embodiments, the mass could be expanded and/or compressed to a shape by natural stresses or forces.

According to various embodiments of the disclosure, the array can be provided pre-made, or standardized, with definite spacing between the seeds. This known array allows calculation of dosimetry to the treated area. The array could be constructed with a standard spacing, or be customized to a seed pattern desired by the end user. For example, the carrier could be provided without radioactive seeds and with provisions in the carrier, such as pouches or slits, to allow for loading of individual seeds by the end user. In this manner, the seeds can be disposed in the carrier with either standardized or customized spacing. A discrete seed spacing could be advantageous in that the end user would not have to weave a suture containing seeds into a mesh. This could provide a time savings, and could ensure that the seeds would have a definite spacing (independent of the skill of the end user in weaving a filament of seeds) and provide reproducible and calculable dosimetry.

According to various embodiments, the implant comprises a bioabsorbable or permanent carrier. Alternatively, the implant can comprise both bioabsorbable and permanent components in the carrier. The use of either a bioabsorbable or permanent carrier allows the physician to tailor the mechanical properties of the implant to fit the tumor type or location of the tumor/tumor bed.

There is a variety of radioactive seeds that can be used in accordance with the present disclosure. Suitable non-limiting examples of such seeds include, for example, I¹²⁵, Pd¹⁰³, Cs¹³¹, Au¹⁹⁸, Co⁶⁰, and Ir¹⁹². Those of ordinary skill in the art will appreciate that any seed or radioactive particle capable of providing a therapeutic dose of radiation can be used. Seeds can be made of a number of different materials known to the ordinary practitioner. For example, the seeds can be in the form of a metallic capsule, a polymer, a ceramic, a ribbon, or can be particulate in nature. Any form capable of providing the desired dose of radiation can be used.

The implant can comprise a variety of materials (in addition to the seeds). For example, the seeds can be entrained within a sheet, woven mesh, knitted mesh, felt, polymeric sheet, fabric, etc. According to various embodiments, the seeds are entrained within a non-absorbable mesh. Suitable non-absorbable meshes are well-known, and include those disclosed in, for example, U.S. Pat. No. 6,971,252 (the disclosure of which is incorporated by reference herein). The meshes can be constructed of at least one of polypropylene, polyester, polyurethane, stainless steel, titanium, carbon fiber, nitinol, and other materials. According to various embodiments, the seeds can be entrained in a non-absorbable material, such as a non-absorbable polymeric sheet. Suitable non-limiting examples of polymeric sheets include polyurethane and silicone.

According to various embodiments, the seeds can be entrained within an absorbable mesh, felt, or sheet. Absorbable materials are well-known to those of ordinary skill in the art, and can be constructed of, for example, polydioxanone, polylactide, polyglycolic acid, absorbable polyurethane, and collagen.

The seeds can be disposed in the carrier via a number of different mechanisms. For example, the seeds can be attached to the carrier via adhesives, welding, thermal bonding, sewing, entrainment between two sheets of material, or placement into formed pockets on the material. The entrainment between two sheets of material can be accomplished by heat staking around the seeds to affix the two sheets together. The heat staking technique could be advantageous in that a second chemical and/or material would not necessarily need to be added to affix the seeds, and the tedious task of sewing with radioactive materials could be avoided. The original properties of the entrainment material, most importantly the ability of tissue to grow into the pores of the material, could also be maintained with minimal disruption during the staking process. The seeds can also be affixed to the material by placing the seeds within a holder or shielding apparatus having a feature that mates with a corresponding feature in, or on the opposite side of, the material.

According to various embodiments, the carrier materials can be homogeneous, or can be constructed of layers or areas of dissimilar materials (e.g., a polypropylene mesh welded to a polyester mesh, with the seeds trapped between). A suitable construction can be selected to adjust physical and performance qualities, including but not limited to flexibility, degree of tissue in-growth, tensile or flexural strength, avenues for sterilization or processing, degree of seed retention, visibility by medical imaging modalities, attachment method to tissue or bone, degradation time, adhesion (e.g., provide a fast-absorbing collagen layer on the outside of a slow-absorbing polylactide material) and control of tissue erosion.

The carrier material can incorporate additional elements for a variety of purposes. Suitable non-limiting examples of such elements include fiducial markers for visualization/localizing by medical imaging modalities (ultrasound, fluoroscopy, MRI, CT, etc.); visual markings indicating alignment, seed placement, seed placement distances, and/or tissue attachment points; features to ease assembly and alignment such as “corduroy” type grooves, dimples, and formed depressions; and coatings to increase/reduce adhesion, promote/retard in-growth, cause coagulation of blood, provide tumoricidal activity, increase biocompatibility, reduce microbiological growth, reduce inflammation, deliver analgesics, etc. In addition, the carrier material can have incorporated therein, or attached thereon, features to ease attachment to tissue such as, for example, loops, arms, filaments, sutures, and staples.

According to various embodiments, the implant can comprise a radiation shielding backing material to afford a directional radiation dose. Suitable non-limiting examples of such materials include bismuth- or barium-loaded polymers. This backing material can be in the form of a solid sheet, or have open areas to allow selective dose transmission. Such a backing material could be useful to direct the dose towards areas of interest while shielding healthy or sensitive tissues or organs. The backing material can be permanent (e.g., bismuth-loaded silicone), removable or bioabsorbable.

Radioactive seeds, such as the BrachySource seeds sold by Bard Brachytherapy (Carol Stream, Ill.) provide a symmetrical “4π” does distribution around the seed. Such seeds can be useful for treating various types of tumors such as prostate tumors, where a tumoricidal dose is desired all the way around a seed.

There are various clinical situations where a limited directional dose or field is desired. For example, to implant seeds in an excised tumor bed following a radical prostatectomy, the clinician would want to irradiate the tumor bed but may not want to irradiate the rectum or bladder. With known seed designs and deployment, this could be difficult because seeds are typically free to rotate or shift from their implanted position. If the seeds were attached to a mesh as described above, the seeds would be fixed and the placement of the mesh would necessarily fix the placement and orientation of the seeds. The radiation field of the seed could then be altered to provide a uni-directional dose.

According to various embodiments, there can be advantages associated with modifying the radiation field surrounding the seed without modifying the seed itself. For example, the modification could potentially be used with any seed in the marketplace; a seed manufacturer would not have to keep as extensive an inventory of different seeds; and the proven structural integrity of a given seed would be unchanged. Several different dose profiles could be offered by merely modifying the exterior shielding apparatus. The shielding method could aid in fixing the seed in the body to confirm the direction of the dose in relation to body structures. In addition, the shielding apparatus could offer a visual verification of the dose direction (which wouldn't necessarily be available with an internal dose modification).

According to various embodiments, there is provided herein radioactive seeds that incorporate a shielding component in one or more desired directions. The shielding can be provided by any biocompatible material such as stainless steel, titanium, tungsten, gold, platinum, etc., with the exact material being selected based on the desired degree of shielding.

The shielding material can be homogenous, or can comprise a plurality of layers (e.g., spring steel electroplated with gold). The layers can be selected in order to modify at least one of biocompatibility, manufacturability, cost, radiopacity, susceptibility to galvanic corrosion, functionality, and durability.

According to various embodiments, the shielding apparatus can be fixed to the seed as a flat plate or as a conformal structure (e.g., a piece of foil could surround a portion of the diameter of the seed). The shielding apparatus can be attached to the seed by any suitable attachment method such as, for example, adhesive, welding (laser, resistance, etc.), soldering, mechanical entrapment, electroplating, etc. According to various embodiments, mechanical entrapment can be accomplished by at least one of a feature that allows a seed to be “snapped” in place; a feature that has seed placement in the shielding followed by crimping, forming, etc., to complete the entrapment; a feature that naturally confirms around the seed at body temperature (e.g., construct the shielding material from nitinol, which is a flat sheet at cool temperatures but which wraps around the seed when warmed to room or body temperature).

The shielding apparatus can include at least one feature that prevents, or at least reduces, rotation or movement of the seed relative to the mesh. For example, the shielding material can have flat areas extending from the seed assembly. The material can comprise barbs or rough areas that grip the mesh structure. According to various embodiments, if the seeds were sewn in place, the shielding material could be provided with ridges, holes, or other features capable of securing the thread.

According to various embodiments, the shielding apparatus could be provided with various agents such as, for example, agents that modify adhesion, promote or retard ingrowth.

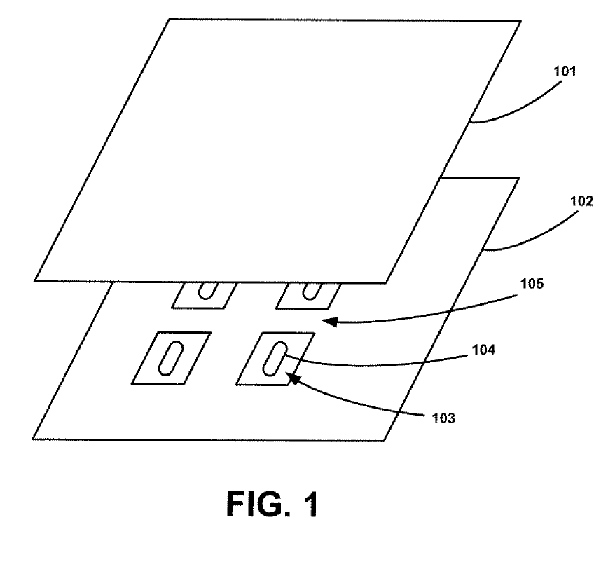

The above-described features of the various embodiments of the present method and apparatus are depicted in FIGS. 1-8.

FIG. 1, according to various embodiments, is an illustration of an implant in accordance with the present disclosure. In this embodiment a first sheet of material 101 may be operatively coupled to a second sheet of material 102. The second sheet of material 102 may have formed pockets 103 which hold seeds 103. The first and second sheets of material 101, 102 may be adhered to one another by heat staking around the seeds in areas 105.

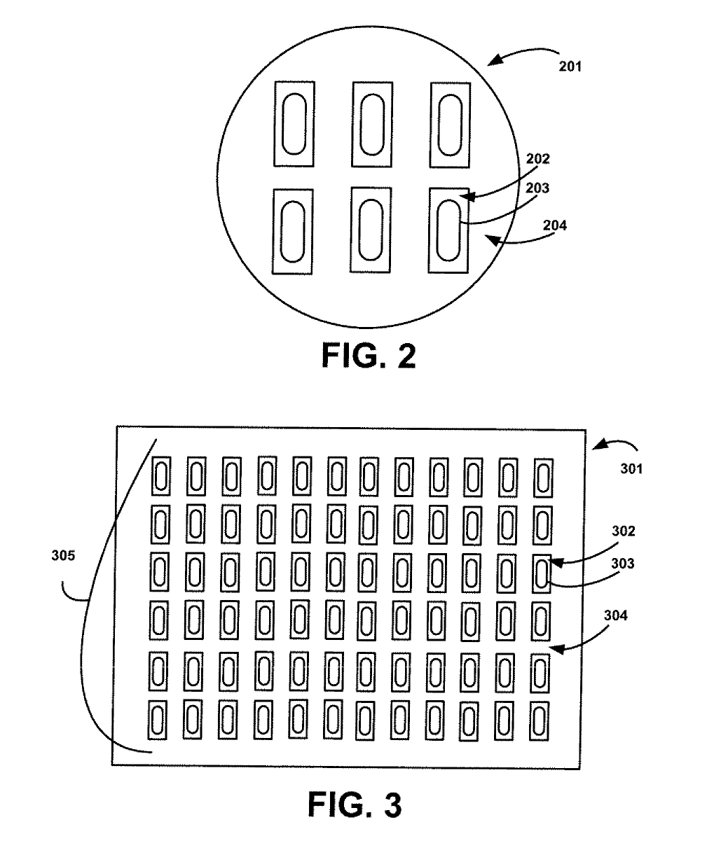

FIG. 2, according to various embodiments, is a detailed illustration of an implant 201 having radioactive seeds 203 in accordance with the present disclosure. The seeds 203 may be held in pockets 202 by staking areas 204. In this embodiment each pocket 202 may be surrounded by one common staking area 204.

FIG. 3, according to various embodiments, is an overall illustration of an implant 301 having radioactive seeds 303 in accordance with the present disclosure. The seeds 303 may be held in pockets 302 by staking area 304. The seeds 303 in the pockets 302 may be arranged in a predetermined pattern 305. The pattern 305 may be constructed with a standard spacing as depicted, or may be customized to a seed pattern desired by the end user.

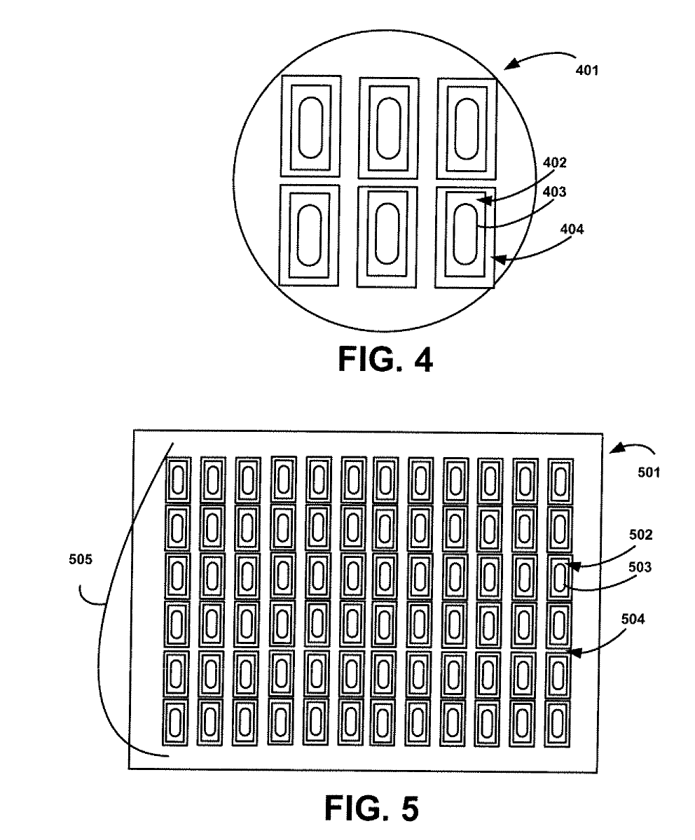

FIG. 4, according to various embodiments, is a detailed illustration of an alternative embodiment of an implant 401 having radioactive seeds 403 in accordance with the present disclosure. The seeds 403 may be held in pockets 402 by staking areas 404. In this embodiment each pocket 402 is surrounded by its own respective staking area 404.

FIG. 5, according to various embodiments, is an overall illustration of an alternative embodiment of an implant 501 having radioactive seeds 503 in accordance with the present disclosure. The seeds 503 may be held in pockets 502 by individual staking areas 504. The seeds 503 in the pockets 502 may be arranged in a predetermined pattern 505. The pattern 505 may be constructed with a standard spacing as depicted, or may be customized to a seed pattern desired by the end user.

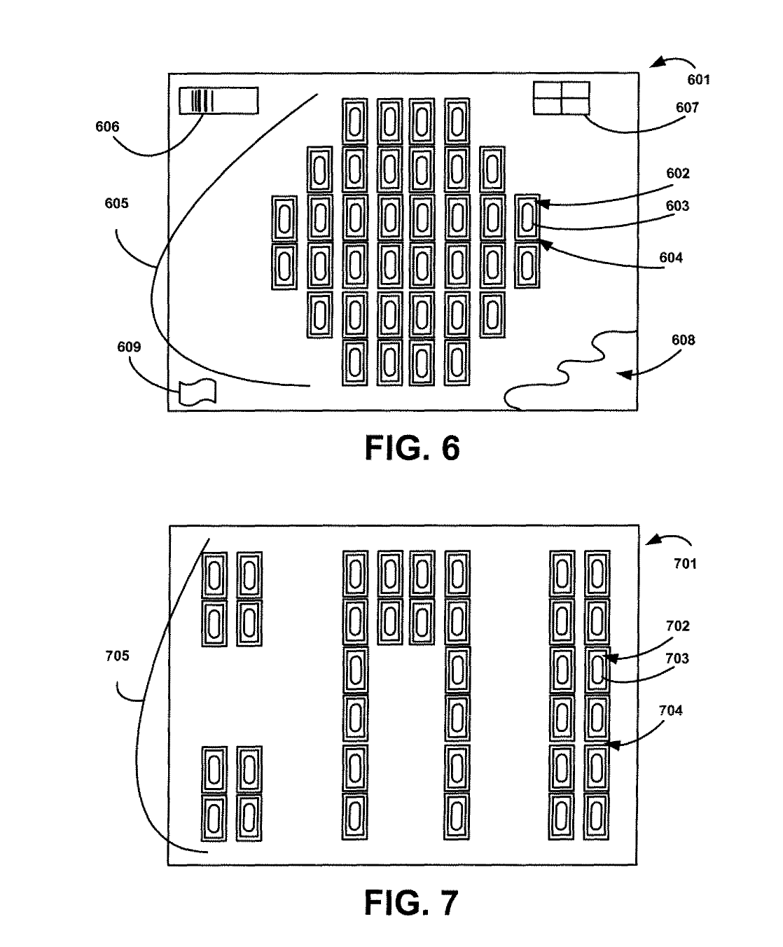

FIG. 6, according to various embodiments, is an overall illustration of an implant 601 having radioactive seeds 603 in a predetermined pattern 605 in accordance with the present disclosure. The seeds 603 may be held in pockets 602 by individual staking areas 604. The implant 601 may incorporate additional elements for a variety of purposes, such as, fiducial markers 606, visual markings 607, coatings 608, and attachment elements 609.

FIG. 7, according to various embodiments, is an overall illustration of an implant 701 having radioactive seeds 703 in another predetermined pattern 705 in accordance with the present disclosure. The seeds 703 may be held in pockets 702 by individual staking areas 704.

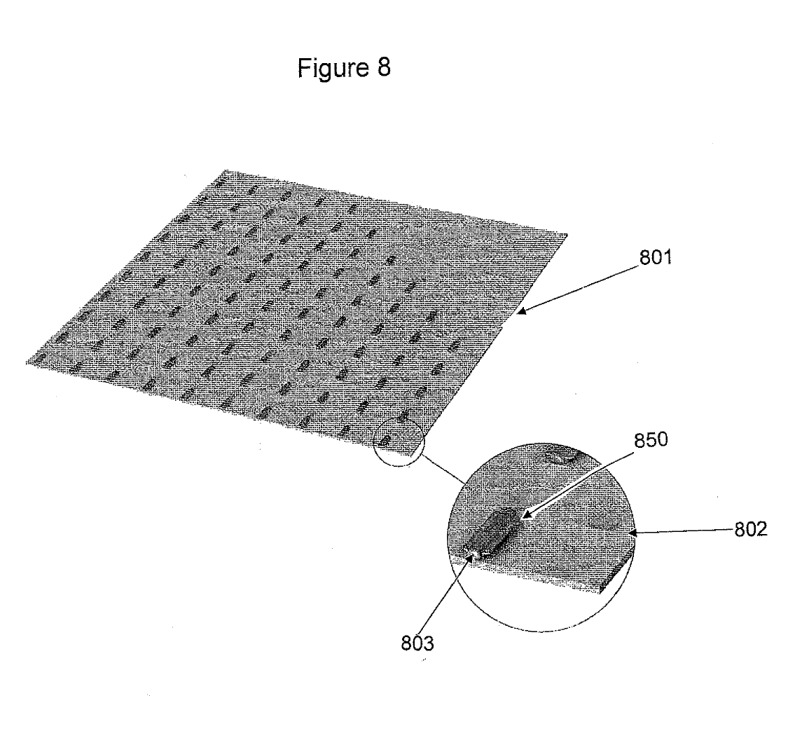

FIG. 8, according to various embodiments, is an overall illustration of an implant 801 having radioactive seeds 803 in a predetermined pattern in accordance with the present disclosure. The seeds 803 are partially disposed in shielding apparatus 850. Shielding apparatus 850 is attached to biocompatible material 802.

The implants can be attached to tissue using a variety of different methods. For example, the implants can be affixed to tissue via at least one of sutures, staples, tacks, adhesives, physical entrapment (chevrons), or other standard tissue-anchoring means. The implants can be permanently flexible, or can be rigid and formed into particular rigid shapes using heat and/or pressure based on the particular application. The implants could be constructed of materials that would change physical properties when contacted with body fluids or exposed to body temperature.

According to various embodiments, the implants can be applied externally or internally. For example, the implants can be inserted laproscopically or by open surgery. The implants can be used in the body, or externally (i.e., a skin patch). The implant can be inserted into a tumor bed. For example, a tumor can be excised from a body cavity and the implant can be fixed to the locus thereof. The implant can be fixed to the tumor bed by a variety of different methods, including suturing, stapling, and adhesion. In the case of a spherical or semispherical implant, the implant can be inserted into the cavity and permitted to expand, thereby filling at least a portion of the cavity.

According to various embodiments, the implant can be provided simultaneously or sequentially with an adjunctive therapy. For example, the implant can comprise a slow-release chemotherapeutic agent. Such agents and their release profiles are well-known to those of ordinary skill in the art of, inter alia, oncology. In addition, external beam radiation can be provided as an adjunct to seed radiation.

For the purposes of this specification and appended claims, unless otherwise indicated, all numbers used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the” include plural referents unless expressly and unequivocally limited to one referent. Thus, for example, reference to “a mesh” includes two or more meshes.

Other various embodiments of the invention will be apparent to those skilled in the art from consideration of the specification and practice of the invention disclosed herein.

It is intended that the specification and examples be considered as exemplary only, with a true scope and spirit of the invention being indicated by the following claims.

1. An implant comprising:
at least one sheet of a biocompatible material;
at least one shielding apparatus fixed to the biocompatible material; and
at least one radioactive seed partially disposed in the shielding apparatus.

2. The implant according to claim 1, comprising a plurality of radioactive seeds.

3. The implant according to claim 1, wherein the radioactive seed is at least substantially fixed into a position by the shielding material.

4. The implant according to claim 1, wherein two sheets of a biocompatible material are bonded together at a perimeter around said at least one radioactive seed, and the at least one radioactive seed is partially disposed in the shielding apparatus.

5. The implant according to claim 1, wherein the shielding apparatus is comprises gold.

6. The implant according to claim 1, wherein at least a portion of the biocompatible material is a bioabsorbable material.

7. The implant according to claim 1, wherein at least one of the biocompatible material and the shielding apparatus comprise at least one of a fiducial marker, a visual marker, a coating, and an attachment feature.

8. The implant according to claim 1, wherein the at least one sheet of a biocompatible material comprises a radiation-shielding material.

9. The implant according to claim 8, wherein the radiation-shielding material is a polymeric material containing at least one of bismuth and barium.

10. The implant according to claim 1, wherein the at least one sheets of biocompatible material is an open-knit mesh.

11. The implant according to claim 10, wherein the open-knit mesh is coated with at least one biological material.

12. The implant according to claim 1, wherein the seeds are disposed in a planar array.

13. The implant according to claim 1, wherein the seeds are disposed in a three-dimensional array in the at least one sheet of biocompatible materials.

14. A method for treating a patient, comprising fixing to the tissue of the patient at least one implant according to claim 1.

15. The method according to claim 14, further comprising treating the patient with at least one additional therapy.

16. The method according to claim 15, wherein the at least one additional therapy is chosen from chemotherapy and external beam radiation.

17. The method according to claim 14, wherein said implant is fixed to the tissue by at least one of sutures, staples, tacks, adhesives, and chevrons.

18. The method according to claim 14, wherein the implant is laproscopically inserted into the patient.
